(19) United States
(12) Patent Application Publication
(10) Pub. No.: US 2024/0285417 A1
(43) Pub. Date: Aug. 29, 2024

(54) TRIAL IMPLANT

(71) Applicant: KUMOVIS GMBH, München (DE)

(72) Inventors: Stefan LEONHARDT, München (DE); Sebastian PAMMER, München (DE)

(21) Appl. No.: 18/573,380

(22) PCT Filed: Jun. 24, 2022
(86) PCT No.: PCT/EP2022/067297

§ 371 Date: Dec. 21, 2023

(30) Foreign Application Priority Data
Jun. 25, 2021 (DE) 10 2021 116 548.2

(51) Int. Cl. A61F 2/46; A61F 2/30

(52) U.S. Cl. CPC A61F 2/4684; A61F 2002/30985

(57) ABSTRACT

The present invention relates to a trial implant, in particular for spinal surgery, a total knee or hip endoprosthesis or a shoulder prosthesis, with at least one head, with at least one reinforcing core and with at least one stem, wherein the head is produced by means of 3D printing, and wherein the head and the stem are made of plastic.

The present invention relates to a trial implant, for example for spinal surgery, for total knee or hip endoprostheses or shoulder prostheses.

In the case of degenerative lumbar and lumbosacral diseases, such as degenerative disc disease, spinal instability or spondylolisthesis, spinal segments are surgically stiffened by removing the disc in the affected segment and then inserting an implant, also known as a cage, into the resulting intervertebral space. To insert the cage, it is first necessary to determine the patient's individual dimensions between the two affected vertebral bodies, as the largest possible cage for fusing the two vertebral bodies should preferably be implanted for reasons of stability. Trial implants, which are inserted into the intervertebral space during the operation, are used to determine the required implant dimensions. Different trial implants can therefore be used to successively determine the best possible dimensions of the cage to be used. The trial implants themselves must have a certain strength and shape to ensure that the two affected, adjacent vertebral bodies are not (further) impaired during insertion into the intervertebral space, alignment in the intervertebral space and final removal from the intervertebral space. For safe handling of the trial implant during the determination of the intervertebral space dimensions, an elongated handling section or shaft is usually provided on the trial implant, whereby this shaft must have sufficient elongation, shear, bending and torsional rigidity. The entire trial implant, i.e. including the stem, is usually manufactured as a unit using an ablative manufacturing process. The ablative production of a trial implant from solid material is known to be costly in terms of production time and production steps and is also not very resource-efficient.

Such trial implants are known, for example, from DE 11 2021 001 452 T5 or EP 1 648 351 B1.

A modular trial implant system is also known from DE 10 2008 030 260 A1. According to this, a trial implant system is of modular design, wherein the at least one trial implant comprises at least one first and at least one second trial implant part, wherein the at least one first and second trial implant parts can be connected to one another in different connection positions relative to one another.

It is therefore the task of the present invention to further develop a trial implant of the type mentioned above in an advantageous manner, in particular in such a way that the trial implants can be individualized more quickly and easily and can be manufactured more cost-effectively.

This task is solved according to the invention by a trial implant with the features of claim 1, according to which it is provided that a trial implant, in particular for spinal surgery, for total knee or hip endoprostheses or shoulder prostheses, is provided, with at least one head, with at least one reinforcing core and with at least one shaft, wherein the head is produced by means of 3D printing, and wherein the head and the shaft are made of plastic.

The invention is based on the basic idea that an easy-to-fabricate trial implant consisting only of the elements head, shaft and reinforcing core is provided, whereby at least the head of the trial implant, which is inserted into the intervertebral space to determine the suitable cage, is produced by means of 3D printing. The 3D printing of the head enables a cost-effective and fast production of an individual trial implant, whereby individual anatomical conditions of the affected vertebral bodies can be easily taken into account. The head can thus be individualized using 3D printing. This means that the dimensions of the head can be adapted to the patient's individual circumstances. The trial implant can be available in standardized sizes, such as “small” (S), “medium” (M) or “large” (L), or can be created directly using rapid prototyping based on the evaluation of preliminary imaging procedures. The reinforcement core of the trial implant is arranged completely within the composite of head and shaft and ensures at least sufficient bending and torsional rigidity of the trial implant. The head preferably has an elongated, stepped section for connection to the shaft and for receiving the reinforcement core. The multi-part design also allows the stem and reinforcement core to be combined with different heads. The shaft can also be manufactured using 3D printing or conventional processes such as turning, injection molding or similar. The material for the head and the shaft can be polypropylene (PP) in the case of a single use of a trial implant and polyphenylsulfonc (PPSU) in the case of a desired multiple use of the trial implant, whereby polyetheretherketone (PEEK), polyetherketoneketone (PEKK) or another polyetherketone are also conceivable for the latter use.

In one possible example embodiment, the reinforcement core consists of a reinforced material and/or a composite material. This means that the choice of material for the reinforcement core can already achieve an advantageous rigidity for the reinforcement core. The reinforcement by e.g. fibers in the material can also have an orientation such as a longitudinal orientation, so that the reinforcement is more pronounced in a certain direction such as the longitudinal direction than in another direction of the reinforcement core.

Possible materials for the reinforcement core can be metallic materials such as titanium alloys or stainless steels, but carbon fiber reinforced plastics (CFRP), such as carbon fiber reinforced polyether ether ketone (CF/PEEK), can also be used. A composite material, such as a metal matrix composite, is also conceivable.

In another possible example embodiment, the material of the reinforcement core consists at least partially of CFRP and/or at least partially of metal. This enables good to very good rigidity, which is particularly appropriate and adapted for use as a spinal implant, even with low material usage and small installation space.

In another possible example embodiment, both the head and the shaft are provided with a recess so that the reinforcement core is completely accommodated by both the head and the shaft when the trial implant is mounted. This prevents the reinforcement core from coming into direct contact with patient tissue, for example. Both the head and shaft can then be provided with a biocompatible material or consist of a biocompatible material, e.g. without fiber reinforcement and at least with their (complete) outer side or the surfaces with which they come into contact with tissue.

The head and shaft can be pushed onto the reinforcement core through the recesses. The recess in the head is preferably provided in its offset section. The total longitudinal extent of the recesses in the head and shaft corresponds to the longitudinal extent of the reinforcement core, so that the latter is accommodated in the trial implant without play when it is mounted, i.e. when the head and shaft are connected.

Both the recess in the head and in the shank can have at least a short cross-sectional taper in relation to the longitudinal expansion, so that the head and the shank are secured against slipping off the reinforcing core regardless of their actual connection.

In another possible example embodiment, the head and shaft completely accommodate and cover the reinforcement core when mounted, so that the reinforcement core is completely concealed.

In the assembled state, the reinforcement core is arranged completely inside the composite of head and shaft so that the reinforcement core does not come into contact with the patient's tissue.

In another possible example embodiment, the head and shaft are connected to each other by means of an adhesive process and/or joining process.

In addition to the possibility of gluing the head and the shaft together at their contact surfaces after insertion of the reinforcement core or after sliding onto the reinforcement core, it is also conceivable to join the two components by means of plastic welding. Furthermore, it is also conceivable that either the head or the shaft has a stepped sleeve, i.e. a sleeve with a smaller outer circumference than the stepped section of the head or shaft, in the connection area, which is dimensioned so that it is pressed into the opposite recess between the reinforcement core and the head or shaft when the head and shaft are pushed together along the reinforcement core.

In a further example embodiment, the reinforcing core has a geometry that secures against rotation relative to the head and/or the shaft.

A polygonal shape in relation to the circumference of the reinforcement core or the circumference of the recess in the head and shaft, such as a triangular or square shape, prevents the head and shaft from twisting relative to each other after being pushed onto the reinforcement core, allowing the trial implant to be aligned in the intervertebral space without interference.

It is also conceivable to provide one or more nubs or ribs or the like.

In a further possible example embodiment, a contrast agent is contained in at least one of the elements head and/or reinforcing core and/or shaft. The contrast agent allows the corresponding element(s) to be better visualized in an imaging procedure, such as X-ray diagnostics, magnetic resonance imaging or sonography, which enables the treating surgeon to optimally align the trial implant in the affected intervertebral space and to determine the required dimensions of the cage to be used. The element provided with a contrast agent can also be better visualized for use in other surgical areas, for example when using the trial implant for a total knee or hip endoprosthesis or for a shoulder prosthesis. The contrast agent is bound in the corresponding element.

In a further possible example embodiment, the contrast agent consists at least partially of barium sulphate. Barium sulphate as a component of the contrast agent is a non-toxic barium compound which, due to its impermeability to X-rays, is preferably used as an X-ray-positive contrast agent in X-ray diagnostics.

In another possible example embodiment, the trial implant has a color code. The color code of the trial implant allows the dimensions or other characteristics of the trial implant, such as the material of the trial implant or the stiffness of the trial implant, to be quickly determined visually during the operation.

It is also conceivable that the head and shaft have different color codes. Accordingly, the color coding of the head can indicate its height, whereby the height of the head is to be understood here as its extension from vertebral body to vertebral body, while the color coding of the shaft can indicate its length. Similarly, the color coding can also be assigned to different dimensions and/or geometries of the head, for example for a total knee or hip endoprosthesis or for a shoulder prosthesis.

Embodiments of the invention are now described below with reference to the drawings. These are not necessarily intended to show the embodiments to scale; rather, where this is useful for explanation, the drawings are shown in schematized and/or slightly distorted form. Reference is made to the relevant state of the art with regard to additions to the teachings directly recognizable from the drawings. It should be noted that various modifications and changes can be made to the shape and detail of an embodiment without departing from the general idea of the invention. The features of the invention disclosed in the description, in the drawings and in the claims may be essential for the further development of the invention, either individually or in any combination. In addition, all combinations of at least two of the features disclosed in the description, the drawings and/or the claims fall within the scope of the invention. The general idea of the invention is not limited to the exact form or detail of the preferred embodiments shown and described below, or limited to any subject matter that would be limited as compared to the subject matter claimed in the claims. In the case of stated design ranges, values lying within the stated limits are also to be disclosed as limit values and can be used and claimed as desired. For the sake of simplicity, the same reference signs are used below for identical or similar parts or parts with identical or similar functions.

Further advantages, features and details of the invention will become apparent from the following description of the preferred embodiments and from the drawings, which show in:

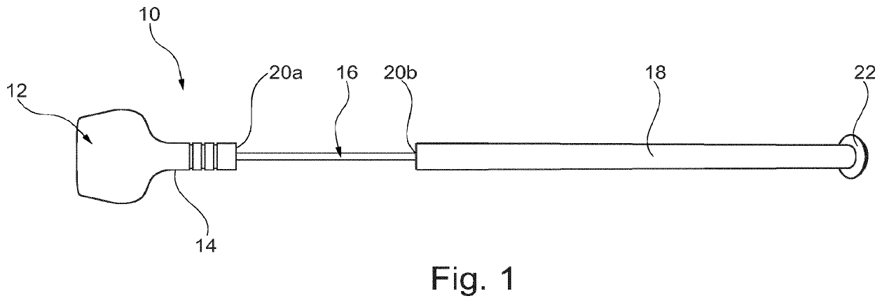

FIG. 1 an example embodiment of a trial implant according to the invention in a pre-assembled state;

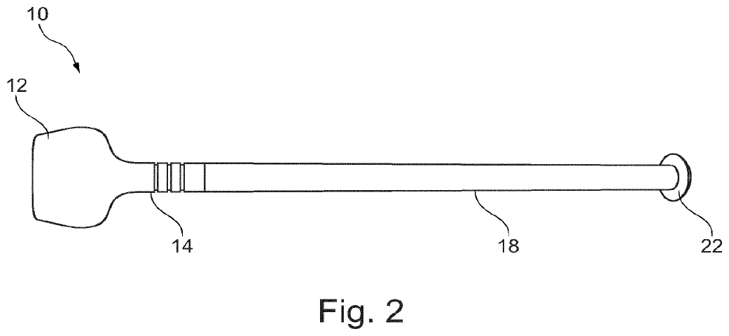

FIG. 2 the example embodiment from FIG. 1 of a trial implant according to the invention in the assembled state;

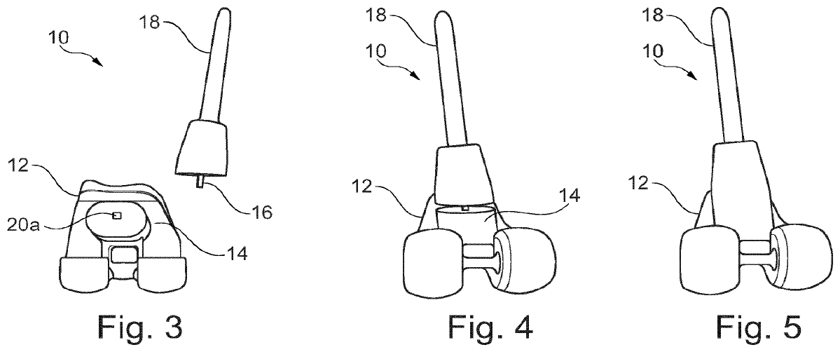

FIG. 3 a further example embodiment of a trial implant according to the invention in a pre-assembled state;

FIG. 4 the example embodiment from FIG. 3 in a further pre-assembled state; and

FIG. 5 the example embodiment from FIG. 3 in the assembled state.

FIG. 1 shows an example embodiment of a trial implant 10 for spinal surgery according to the invention in a pre-assembled state.

The trial implant 10 comprises at least one head 12, at least one reinforcement core 16 and at least one shaft 18.

The head 12 comprises a stepped section 14 and a first recess 20a.

The stepped section 14 of the head 12 and the shaft 18 have a round cross-section (i.e. the cross-section is perpendicular to the longitudinal axis of the implant).

The outer edges of head 12 are rounded.

The shaft 18 comprises a second recess 20b and connection section 22.

The recesses 20a, 20b are only indicated on the input side in FIG. 1, as these are arranged completely within the head 12 or the shaft 18.

The reinforcing core 16 has a square shape. In particular, the cross-section perpendicular to the longitudinal axis of the reinforcing core 16 is square.

The circumferential shape of the recesses 20a, 20b corresponds to the circumferential shape of the reinforcing core 16.

The common length of the recesses 20a, 20b in the head 12 and shaft 18 corresponds to the longitudinal extension of the reinforcing core 16.

As a result, the reinforcement core 16 is completely accommodated by the head 12 and shaft 18 in the assembled state and covered by these, as shown in FIG. 2.

The square shape of the reinforcing core 16 prevents the head 12 and the shaft 18 from rotating around the longitudinal axis and thus also relative to each other.

At least the head 12 was individualized using 3D printing, i.e. adapted to the conditions of the affected intervertebral space of the patient to be treated.

The shaft 18 can also be manufactured using 3D printing; however, it is also conceivable that it was manufactured from solid material using a machining manufacturing process, such as turning.

The head 12 and the shaft 18 are made of plastic.

The round shape of the stepped section 14 of the head 12 and the shaft 18 as well as the rounded edges of the head 12 ensure that no tissue and/or the vertebral bodies are impaired or injured during insertion and removal of the trial implant 10.

The head 12 is used to measure the patient's intervertebral space in order to determine the dimensions of the cage to be inserted. The shaft 18 enables insertion, positioning and removal.

Further instruments for holding and/or guiding the trial implant 10 can be arranged or attached to the connection section 22 of the shaft 18.

The head 12 and shaft 18 can be made of the same or different materials. The material for the head 12 and the shaft 18 can be polypropylene (PP) in the case of a single use of the trial implant 10 and polyphenylsulfone (PPSU) in the case of a multiple use of the trial implant 10, whereby various polyetherketones are conceivable in the latter case.

Metallic materials such as titanium alloys or stainless steels as well as carbon fiber reinforced plastics (CFRP) can be used for the reinforcement core 16; the use of a composite material, such as a metal matrix composite, is also possible.

In FIG. 2, the stepped section 14 of the head 12 and the shaft 18 are connected to each other when the trial implant is mounted.

The contact surfaces of the stepped section 14 and the shaft 18 can be bonded. However, it is possible for the stepped section 14 and the shaft 18 to be joined together by means of plastic welding.

Both head 12 and/or shaft 18 contain a contrast agent, in this case barium sulfate. The contrast agent allows the corresponding element(s) to be better visualized in an imaging procedure, such as X-ray diagnostics, magnetic resonance imaging or sonography, which enables the treating surgeon to optimally align the trial implant in the affected intervertebral space and to determine the required dimensions of the cage to be inserted. The contrast agent is appropriately mixed into the respective material of the head 12 and the shaft 18 during manufacture. This is particularly easy because the head 12 and possibly also the stem 18 are manufactured using 3D printing.

The trial implant also has a color code, here white for the head with the “large” version. Other sizes can be visualized with additional colors such as red for medium size and blue for small size. In principle, it is possible to define the color code and the associated sizes as required. The color code of the trial implant can also be used to quickly visually record the dimensions or other characteristics of the trial implant, such as the material of the trial implant or the stiffness of the trial implant, during the operation.

It is also conceivable that the head and shaft have different color codes. Accordingly, the color coding of the head can indicate its height, whereby the height of the head is to be understood here as its extension from vertebral body to vertebral body, while the color coding of the shaft can indicate its length.

FIG. 3 shows an example embodiment of a trial implant 10 for a total knee endoprosthesis according to the invention in a pre-assembled state.

The trial implant 10 is essentially analogous to the example embodiment described above with reference to FIGS. 1 and 2. Only the differences are therefore described in more detail below.

Compared to the example embodiment shown above with reference to FIG. 1, the shape of the head 12 is adapted to the application for use for a total knee endoprosthesis.

In this example embodiment, the trial implant 10 also comprises at least one head 12, at least one reinforcing core 16 and at least one shaft 18.

The head 12 has a stepped section 14.

In FIG. 3, too, the recesses 20a, 20b of the head 12 or the shaft 18 are only indicated on the input side, as these are arranged completely within the head 12 or the shaft 18.

In the pre-assembled state shown in FIG. 4, the reinforcing core 16 is not yet fully accommodated by the head 12 and shaft 18. Instead, a gap can be seen between the head 12 and the shaft 18.

In the assembled state shown in FIG. 5, however, the reinforcing core 16 is completely accommodated by the head 12 and shaft 18 and the shaft 18 is in direct contact with the head 12.

In the example embodiment of FIGS. 3 to 5, at least the head 12 was also individualized by means of 3D printing, i.e. adapted to the conditions of the affected body part of the patient to be treated.

The shaft 18 can also be manufactured using 3D printing; however, it is also conceivable that it was manufactured from solid material using a machining manufacturing process, such as turning.

The head 12 and the shaft 18 are made of plastic.

In particular, the head 12 is used to measure a space in the patient's anatomy in order to determine the dimensions of the implant to be inserted. The shaft 18 enables insertion, positioning and removal.

Metallic materials such as titanium, titanium alloys or stainless steels as well as carbon fiber reinforced plastics (CFRP) can be used for the reinforcement core 16; the use of a composite material, such as a metal matrix composite, is also possible.

### REFERENCE SIGN

10 Trial implant for spinal surgery
12 Head
14 Stepped section of the head
16 Reinforcement core
18 Shank
20a. 20b Recess
22 Connection section

1. Trial implant, with at least one head, with at least one reinforcing core and with at least one shaft, wherein the head is produced by means of 3D printing, and wherein the head and the shaft are made of plastic.

2. Trial implant according to claim 1,
wherein
the reinforcing core comprises a reinforced material and/or a composite material.

3. Trial implant according to claim 2,
wherein
the material of the reinforcing core is comprised at least partly of CFRP and/or at least partly of metal.

4. Trial implant according to claim 1,
wherein
both the head and the shaft are provided with a recess so that the reinforcing core is completely accommodated by both the head and the shaft when the trial implant is mounted.

5. Trial implant according to claim 4,
wherein
the head and shaft completely cover the reinforcement core when assembled, so that the reinforcement core is completely concealed.

6. Trial implant according to claim 1,
wherein
the head and shaft are joined together by means of an adhesive process and/or joining process.

7. Trial implant according to claim 1,
wherein
the reinforcing core has a geometry which secures against rotation relative to the head and/or the shaft.

8. Trial implant according to claim 1,
wherein
a contrast agent is contained in at least one of the elements head and/or reinforcing core and/or shaft.

9. Trial implant according to claim 1,
wherein
the contrast medium is comprised at least partly of barium sulphate.

10. Trial implant according to claim 1,
wherein
the trial implant has a color code.

11. Trial implant according to claim 1, wherein the trial implant is for spinal surgery, a total knee or hip endoprosthesis, or a shoulder prosthesis.
